(19) United States
(12) Patent Application Publication
(10) Pub. No.: US 2015/0065472 A1
(43) Pub. Date: Mar. 5, 2015

(54) INTRAUTERINE APPLICATION OF 18-METHYL-15SS,16SS-METHYLENE-19-NOR-20-SPIROX-4-EN-3-ONE SYSTEMS, INTRAUTERINE SYSTEMS CONTAINING 18-METHYL-15SS,16SS-METHYLENE-19-NOR-20-SPIROX-4-EN-3-ONE, AS WELL AS THE USE THEREOF IN CONTRACEPTION AND GYNAECOLOGICAL THERAPY

(71) Applicant: BAYER PHARMA AKTIENGESELLSCHAFT, Berlin (DE)

(72) Inventors: Norbert Schmees, Berlin (DE); Lars Röse, Berlin (DE); Tuula Valo, Turku (FI); Katja Prelle, Ratingen (DE); Reinhard Nubbemeyer, Berlin (DE); Henriikka Korolainen, Loimaa (FI); Harri Jukarainen, Kuusisto (FI)

(73) Assignee: BAYER PHARMA AKTIENGESELLSCHAFT, Berlin (DE)

(21) Appl. No.: 14/396,736

(22) PCT Filed: Apr. 19, 2013
(86) PCT No.: PCT/EP2013/058220

§ 371 Date: Oct. 23, 2014

(30) Foreign Application Priority Data
Apr. 23, 2012 (DE) 102012206652.7

(51) Int. Cl. A61K 31/58; A61K 9/00

(52) U.S. Cl. CPC A61K 31/58; A61K 9/0039
USPC 514/173; 549/331

(57) ABSTRACT

The present invention describes intrauterine use of 18-methyl-15β,16β-methylene-19-nor-20-spirox-4-en-3-ones of the general formula (1)

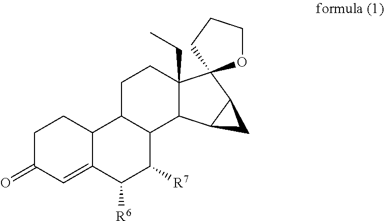

in contraception and gynaecological treatment, and an intrauterine system comprising compounds of formula 1.

The present invention relates to the subject matter characterized in the patent claims, i.e. the intrauterine use of 18-methyl-15β,16β-methylene-19-nor-20-spirox-4-en-3-ones of the formula (I), and to an intrauterine system comprising 18-methyl-15β,16β-methylene-19-nor-20-spirox-4-en-3-ones of the general formula I,

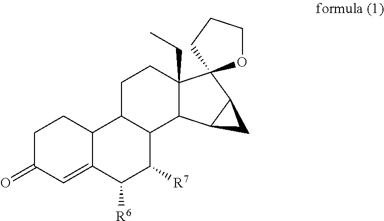

wherein R⁶ and R⁷ may be a hydrogen atom or together may be an α-methylene group.

The invention therefore relates to the intrauterine use of 18-methyl-15β,16β-methylene-19-nor-20-spirox-4-en-3-one (compound A) or 18-methyl-6α,7α,15β,16β-dimethylene-19-nor-20-spirox-4-en-3-one (compound B)

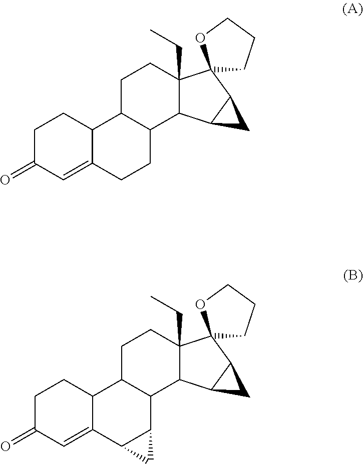

and to an intrauterine system (IUS) comprising one of the said compounds.

The invention further relates to the use of an IUS comprising the substance (A) or (B) in contraception and in gynaecological treatment.

Gynaecological treatment means, for example, treatment of endometriosis, endometrial hyperplasia, inflammation of the endometrium (endometritis), uterine-based pain and dysmenorrhoea, except use in the treatment of menorrhagia [also known as hypermenorrhoea or heavy menstrual bleeding (HMB)] and other forms of uterine bleeding disorders.

The invention also relates to the use of 18-methyl-15β,16β-methylene-19-nor-20-spirox-4-en-3-one (compound A) in contraception and gynaecological treatment.

The progestins employable according to the invention, 18-methyl-15β,16β-methylene-19-nor-20-spirox-4-en-3-one (A) or 18-methyl-6α,7α,15β,16β-dimethylene-19-nor-20-spirox-4-en-3-one (B), and preparation thereof, are described in WO 2008/000521, with the former compound (A) being disclosed there only as intermediate.

Compound B and further substances described in WO 2008/000521, are used in pharmaceutical preparations for contraception and in the therapeutical treatment of premenstrual complaints such as headaches, depressive moods, water retention and mastodynia. WO2008/000521 discloses in addition to oral and transdermal dosage forms also parenteral oily injection solutions. However, WO 2008/000521 does not describe an intrauterine use, nor the compounds being employed in an intrauterine system (IUS).

Approaches with hormone-based contraceptives are widely accepted by users owing to easy application and high contraceptive reliability. Among them, oral contraceptives (pill) are the most frequently used contraceptive method of all in many countries. Nevertheless, there is also time and again a critical discussion of hormone-containing, more specifically oestrogen-containing, contraceptives taking place both publicly and in the literature, owing to potential risks (such as a slightly increased risk of thrombosis, loss of libido, nausea, and headache)¹. ¹ Bitzer et al. Contraception 84 (2011) 342-356

A promising new method of contraception involves the intrauterine administration of hormones by means of an appropriate intrauterine system. At the forefront is Mirena®, a levonorgestrel-containing intrauterine system (IUS) which continuously releases the active ingredient over a period of up to five years. The product is used in contraception and in the treatment of increased menstrual bleeding (menorrhagia or hypermenorrhoea). This product is described in, inter alia, EP 0652738 B1 and EP 0652737 B1.

The basis of the contraceptive effect of Mirena® is essentially the thickening of the cervical mucus and the local action of levonorgestrel which results in a strong anti-proliferative effect on the endometrium. Furthermore, levonorgestrel alters the utero-tubal environment and impairs the motility and function of sperm.

Although the contraceptive effect of Mirena® is mainly a result of a local effect, the comparatively high systemic stability of levonorgestrel (active ingredient in Mirena®) means that Mirena® also exhibits plasma levels of active ingredient of on average about 206 pg/m1². Although this value is below that of orally administered levonorgestrel-containing contraceptives, it is still high enough for it to inhibit ovulation in about 20% of users in the first year of use and for it to be able to cause the known systemic adverse events, for example acne, depressed moods, chest pain or reduced libido³. ² See information sheet Mirena March 2011 DE/9³ Lahteenmäki P. et al. Steroids 2000 65: 693-697

Some first-time users also have a problem with irregular bleeding patterns in the initial phase, i.e. immediately after inserting the IUS (so called “spotting”). This spotting may last for up to a few months before either no or only very small and infrequent haemorrhages occur⁴. ⁴ Suvisaari J, Lähteenmaki P.-Contraception 1996 Oct; 54(4): 207-8

Benign ovarian cysts are described as a further common adverse event of Mirena®⁵. ⁵ Product monograph—Mirena 8th edition August 2009; Finland: Schering AG and Leiras Oy,

Besides the aforementioned hormonal (contraceptive) methods, there is a broad range of non-hormone-based approaches and products, such as natural contraceptive methods (e.g. hormone level measurement, or temperature method), mechanical methods (e.g. condom or diaphragm) or chemical methods (e.g. spermicides). Unfortunately, none of the alternative methods (apart from irreversible sterilization) provides contraception with close to the same level of reliability as achieved by hormone-based methods.

It is therefore an object of the present invention to provide a contraceptive method which not only provides a comparatively high level of reliability in contraception as achieved by the known hormone-based contraceptive methods, but also exhibits even better compatibility.

Another object of the invention is to achieve a regular bleeding pattern, i.e. less spotting, more quickly⁶.

⁶ Andersson et al. Contraception 1994, 49:56-71

The object is achieved according to the invention by the intrauterine use of compounds of formula (I)

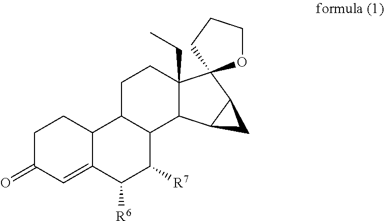

wherein R⁶ and R⁷ are a hydrogen atom or together are an α-methylene group, namely by the intrauterine use of 18-methyl-15β,16β-methylene-19-nor-20-spirox-4-en-3-one or 18-methyl-6α,7α, 15β,16β-dimethylene-19-nor-20-spirox-4-en-3-one.

Surprisingly in the intrauterine use of 18-methyl-15β,16β-methylene-19-nor-20-spirox-4-en-3-one or 18-methyl-6α,7α,15β,16β-bis-methylene-19-nor-20-spirox-4-en-3-one in rats, we were able to demonstrate a differentiating action between local (uterus) and systemic (peripheral tissue) effects.

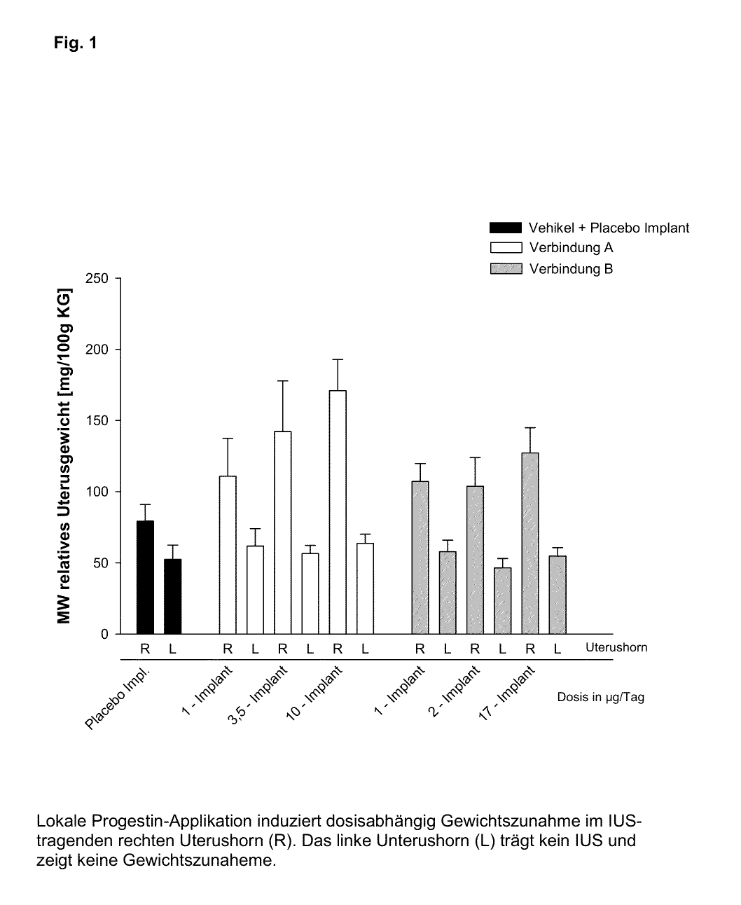

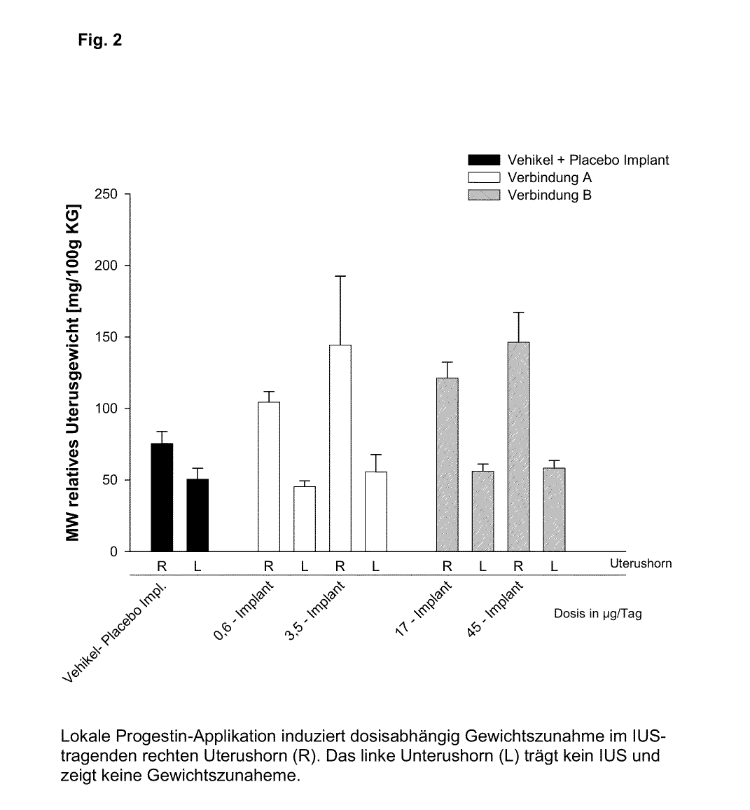

This effect was demonstrated by comparing the local effects in the uterus (weight increase, see Example 1; FIG. 1/4) and by the systemic effect such as, for example, lowering of the LH level in ovary-resected rats (FIG. 2/4).

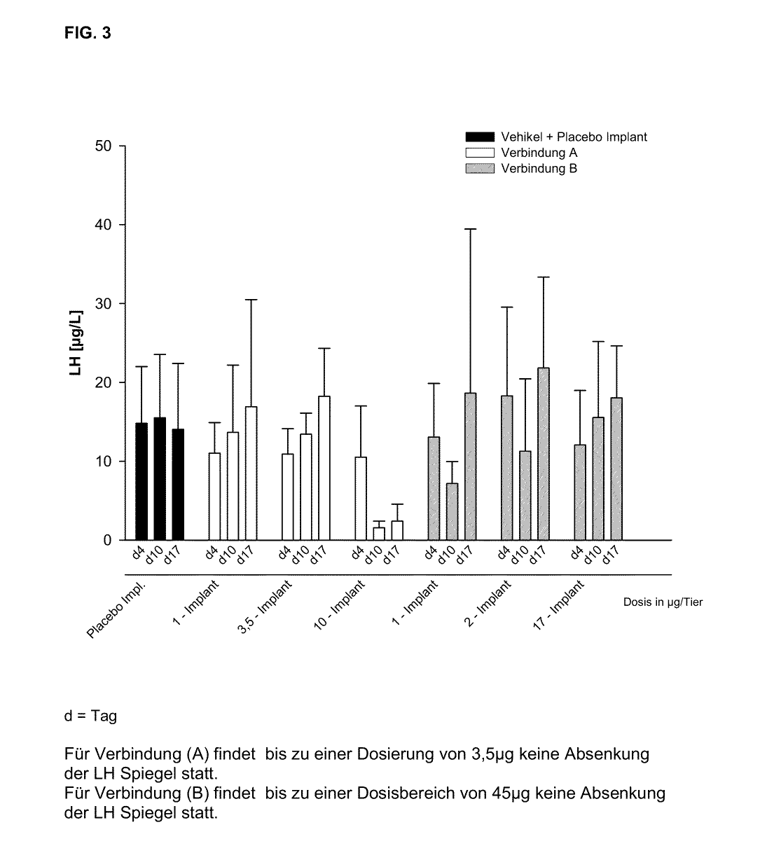

Compared with LNG, the substances also have increased local potency, as shown by the strong induction of corresponding marker genes in the gene expression experiment. Thus, the anti-oestogenic effect of gestagens on the uterus is mediated inter alia by IGFBP-1. FIG. 3/4 shows that IGFBP-1 gene expression is induced by compound A, even at a rate of release from the IUS that is approx. 7× lower than with levonorgestrel.

The marked strong dissociation of local vs. systemic and the high gestagenic efficacy of the substances are sufficient for causing a contraceptive action only due to the local effects. Systemically caused side effects, such as those occurring with the use of other gestagens, may thus be prevented or at least greatly reduced. Owing to the possible higher local gestagen concentration, a more rapidly commencing and better bleeding control can also be expected.

As furthermore demonstrated in comparative transactivation studies (see Example 2), the substances employed according to the invention have an androgenic effect that is at least 10 times lower compared to LNG. This property, still enhanced by the marked dissociation of local vs. systemic, shows that, even with local uterine uses of very high doses in comparison with levonorgestrel, no systemic androgenic effects (e.g. acne) are expected, even if systemic concentrations comparable to levonorgestrel with Mirena® uses were present.

Owing to the properties mentioned of substances A and B, the latter are very well suited to intrauterine use in gynaecological treatment, but in particular also in contraception. Preference is given here to an intrauterine administration by means of an intrauterine system.

An intrauterine system which may be utilized is a polymer system, as is employed, for example, with Mirena®.

A person skilled in the art is familiar with the preparation of an IUS which is carried out as described, for example in EP 0 652 738 B1.

Thus the active ingredient (A) or (B) is first made with a polymeric support material into a central rod (core). The active ingredient may be admixed with the polymeric support material, for example polydimethylsiloxane (PDMS), at any ratio.

After the shaping process, i.e. after vulcanization, the core prepared in this way is normally surrounded in a second step by a polymer-based membrane which ensures uniform dosing over a long period. The desired release rate can be controlled via the choice of polymer and via the thickness of the membrane.

Suitable polymers for the membrane are in principle the same polymers as those for the core (the central rod). Mention must be made here, for example, of polydimethylsiloxane which may optionally be fluorinated, or else other mixtures of polymers. Membrane thickness is preferably around 0.5 mm.

The membrane is applied by firstly swelling a tubing (membrane) prepared from the desired polymer in a solvent and then pressing the core containing the active ingredient into the still swollen tubing. The ends of the tubing are then preferably also sealed by a stopper, preferably consisting of the same material as the tubing/membrane, in order to counteract “bleeding” of the active ingredient at the ends of the tubing, which may result in a “burst effect” during use. The tubing may also be bonded with silicone in place of the stoppers.

Systems releasing a daily dose in the range of 1-500 μg of the particular active ingredient (A) or (B) may be employed according to the invention.

The release rate of active ingredient (A) may be chosen here to be half of that of active ingredient (B), owing to the higher efficacy of the former.

Thus, the resulting preferred dose range for the active ingredient (A) is 1-200 μg/day, with particular preference being given to 1-100 μg/day in particular 2-50 μg/day. The preferred dose range of active ingredient (B) is 2-500 μg/day, with particular preference being given to 2-200 μg/day, in particular 5-100 μg/day.

The invention therefore also relates to an intrauterine system comprising the active ingredient (A) or (B), and to the use of the intrauterine system in contraception.

The examples below serve to illustrate the invention.

The progestins employable according to the invention, 18-methyl-15β,16β-methylene-19-nor-20-spirox-4-en-3-one (compound A) or 18-methyl-6α,7α,15β,16β-dimethylene-19-nor-20-spirox-4-en-3-one (compound B), are prepared as described in WO 2008/000521 (compound A: example 14 f; compound B: example 2).

The process of preparing the active ingredient-charged rods used in the rat experiment described below was carried out similarly to the process of preparing the active ingredient reservoirs, as described for an IUS usable in humans, for example (see for example EP 0 652 738 B1). Polymers which may be used for preparing the rod are polyslioxanes and modified polysiloxane polymers (see for example EP 0652738 B1, WO 00129464 and WO 00100550).

Specifically, first an active ingredient-charged core was prepared by vulcanizing a mixture of polyethylene oxide block-polydimethylsiloxane copolymer (PEO-b-PDMS), polydimethyisiloxane and 10 per cent by weight of the active ingredient (in this case the particular progestin A or B), using a Pt (0)-divinyltetramethyldisiloxane catalyst.

It is also possible to use polydimethylsiloxane (PDMS) rather than PEO-b-PDMS, with bis(2,4-dichlorobenzoyl) peroxide having been used here as the vulcanization catalyst.

To prepare the active ingredient-containing core, a vertical piston unit with a corresponding nozzle head was used. The dimensions of the nozzIe head were such that the outer diameter of the active ingredient-containing core is about 1 mm.

The active ingredient-containing core prepared in this way is then coated with a membrane consisting of PDMS, polytrifluoropropylmethylsiloxanes (PTFPMS) or a PTFPMS/PDMS elastomeric mixture (75% PTFPMS, 25% PDMS). The inner diameter of the membrane material was ˜1 mm, with an outer diameter of ˜1.5 mm.

The coating was carried out by cutting the core and the membrane to a length of 10-15 mm, with the membrane being slightly longer (respectively approx. 1 mm at either end) than the core, in order to enable the ends of the membrane to be sealed with a small stopper after the core has been inserted. In order to enable the core to be inserted into the membrane, the latter was first made to swell in a cyclohexane or acetone-hexane mixture. The active ingredient-containing core was then pushed into the swollen membrane. Finally, the ends of the tubing were either bonded with silicone or sealed with a small stopper made of PTFPMS.

### EXAMPLE 1

The local uterine action of the progestin compared to systemic side effects (dissociation) was investigated on the basis of studies using rats. The uterus of ovary-resected rats responds to implantation of progestin-containing IUS (rods) with decidualization and weight gain. The local progestin effects were also determined on the basis of changes in gene expression.

Serum levels of luteinizing hormone (LH) are used for detecting systemic effects of the locally administered progestin. Basal serum-LH levels of ovary-resected rats are elevated compared to the levels of intact control animals. Undesired systemic efficacy of the uterine-administered progestin can be detected by a decrease in the LH level.

Method:

Ovary-resected female rats were treated with estradiol (E2) for three days (0.2 μg/day/animal, subcutaneous dosing). On day 4, an IUS (rod) was implanted into the right uterine horn of each animal. The left uterine horn remained untreated for internal comparison. Administration of E2 was continued with a daily dose of 0.1 μg/animal to ensure responsiveness of the uterus (maintaining progesterone-receptor expression) to progestins. Blood was taken for LH level measurements on days 4, 10 and 17.

Performing the gene expression analyses:

The uterine tissue was homogenized in 800 μl of RLT lysis buffer (Qiagen, Hilden, Germany; #79216) using a Precellys24 homogenizer (Peqlab, Erlangen, Germany; 2.8 mm ceramic beads; #91-PCS-CK28, 2× 6000 rpm). 400 μl of the homogenate obtained were used for isolating total RNA, using the QIAsymphony RNA kit (Qiagen, #931636) on a QIAsymphony SP robot for automated sample preparation. Reverse transcription of from 1 μg to 4 μg of total RNA was carried out using the SuperScript III first-strand synthesis system (Invitrogen, Carlsbad, USA; #18080-051) according to the random hexamer procedure.

Gene expression analysis was carried out with from 50 ng to 200 ng of cDNA per reaction on an SDS7900HT Real.time PCR system (Applied Biosystems, Carlsbad, USA) using TaqMan probes (Applied Biosystems; IGFBP-1 Rn00565713_m1, Cyp26a1 Rn00590308_m1, PPIA Rn00690933_m1) and the Fast Blue qPCR MasterMix Plus (Eurogentec, Liège, Belgium; #RT-QP2X-03+FB). For relative quantification, cyclophilin A (PPIA) was used as an endogenous control. Relative expression levels were calculated according to the comparative delta delta CT method.

Results:

18-Methyl-15β,16β-methylene-19-nor-20-spirox-4-en-3-one (compound A) and 18-methyl-6α,7α,15β,16β-dimethylene-19-nor-20-spirox-4-en-3-one (compound B) exhibited dose-dependent local efficacy by way of weight gain in the IUS-carrying uterine horn (FIG. 1/4).

Within the release range tested (for compound A: 0.6 -10 μg per animal and day, and for compound B: 1-45 μg/animal and day) both progestins surprisingly exhibited no LH decrease and therefore no systemic side effect, with the exception of the 10 μg/animal and day dose of compound A (FIG. 2/4).

The pharmacokinetic profile of 18-methyl-15β,16β-methylene-19-nor-20-spirox-4-en-3-one and 18-methyl-6α,7α,15β,16β-dimethylene-19-nor-20-spirox-4-en-3-one, respectively, indicated a very fast break-down rate in all in-vitro metabolic studies (liver) as well as in all animal species studied in vivo.

With local administration by means of IUS (rods) in rats, compound A exhibited a 4- to 7-fold higher potency in inducing gene expression than levonorgestrel, with identical release rates (FIG. 3/4). This higher local potency additionally supports the possibility of achieving more rapid and stronger local gestagenic effects on the uterus without causing systemic side effects in the process.

As a result, these progestins can be dosed with local efficacy in such a way that the side effects described for levonorgestrel do not occur in the woman.

Likewise, very rapid break-down rates have also been found in vitro (liver) for humans. The rapid in-vitro breakdown in the liver may also indicate rapid in-vivo breakdown, resulting in a very low systemic exposition of 18-methyl-15β,16β-methylene-19-nor-20-spirox-4-en-3-one and 18-Methyl-6α,7α,15β,16β-dimethylene-19-nor-20-spirox-4-en-3-one after administration through an IUS being calculated. The expected substance levels (Css=concentration at steady state) are calculated from the rate of release from the IUS divided by the clearance. Using a dose of 20 μg per day and woman, which corresponds to that of Mirena, gives a calculated systemic exposition (load) for 18-methyl-15β,16β-methylene-19-nor-20-spirox-4-en-3-one and 18-methyl-6α,7α,15β,16β-dimethylene-19-nor-20-spirox-4-en-3-one, which is over 30 times lower in comparison with Mirena®.

### EXAMPLE 2

The action on the human androgen receptor (hAR) was studied by means of transactivation analyses. For this, different concentrations of the test substances were to cells with stable expression of the human androgen receptor, and activation of the androgen receptor can be detected via a reporter gene.

Method:

For transactivation studies, PC3 (human prostate carcinoma) cells which have been stably transfected with hAR and the MTV-luc reporter gene were used. The culture medium used was RPM! medium (without L-glutamine; without Phenol Red) #E15-49 PAA L-glutamine 200 mM #25030-024 Gibco BRL 100 U/100 μg/ml penicillin/streptomycin Gibco # 15140-122, with 10% foetal calf serum (FCS). The cells were cultured at 37° C. and 5% CO2. The test medium corresponded to the culture medium, except that 10% FCS was replaced with 5% activated carbon-treated FCS (CCS). Cells were seeded in wells of a 96 well plate (“CulturPlate” from Packard #6005180) with 2×104 cells/well/200 μl of test medium. The cells were incubated with different concentrations of the test substances, and 80 μl of substrate were measured using the “steadylite HTS Reporter Gene Assay System” from Perkin Elmer.

Results:

The results show that compound A (18-methyl-15β,16β-methylene-19-nor-20-spirox-4-en-3-one) and compound B (18-methyl-6α,7α,15β,16β-bis-methylene-19-nor-20-spirox-4-en-3-one) have an EC50 in hAR transactivation which is more than 10 times higher than that of levonorgestrel: While the EC50 values are 6.9 nM for compound A and 56 nM for compound B, levonorgestrel has an EC50 of only 0.5 nM. This >10-fold dissociation over levonorgestrel means that no systemic androgenic effects are expected when the compounds are used, even if local uterine uses were to produce systemic active ingredient levels as those observed for levonorgestrel with Mirena® uses.

### EXAMPLE 3

The amounts of active ingredient (A) or (B) released were determined by means of reversed-phase liquid chromatography with UV detection in a 1% strength 2-hydroxypropyl-β-cyclodextrin (2-HPBCD) solution.

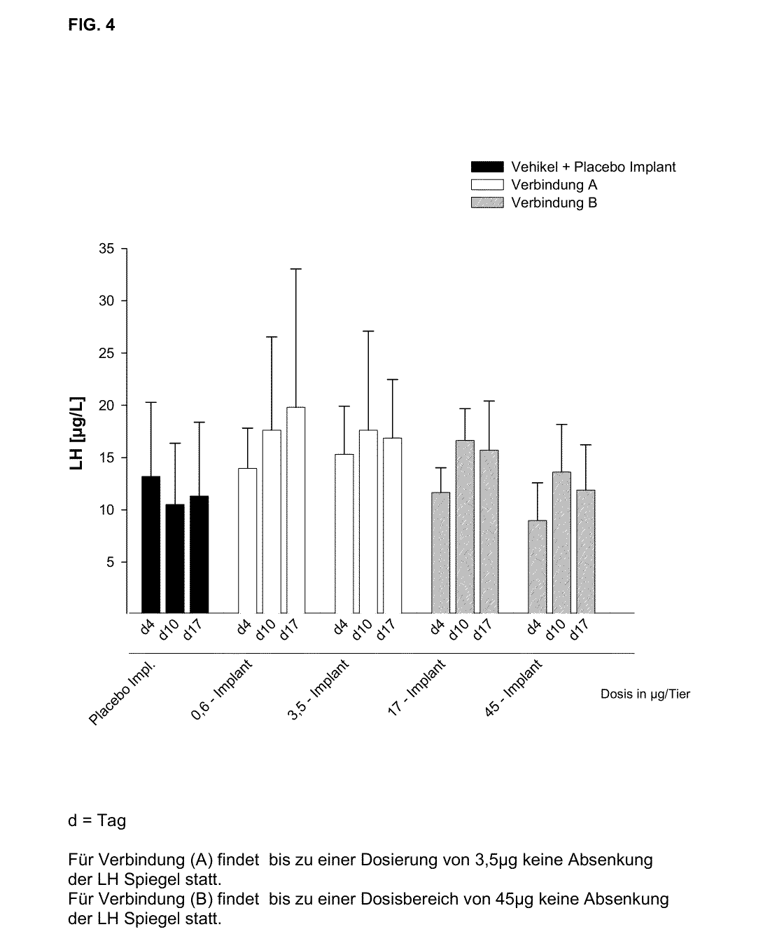

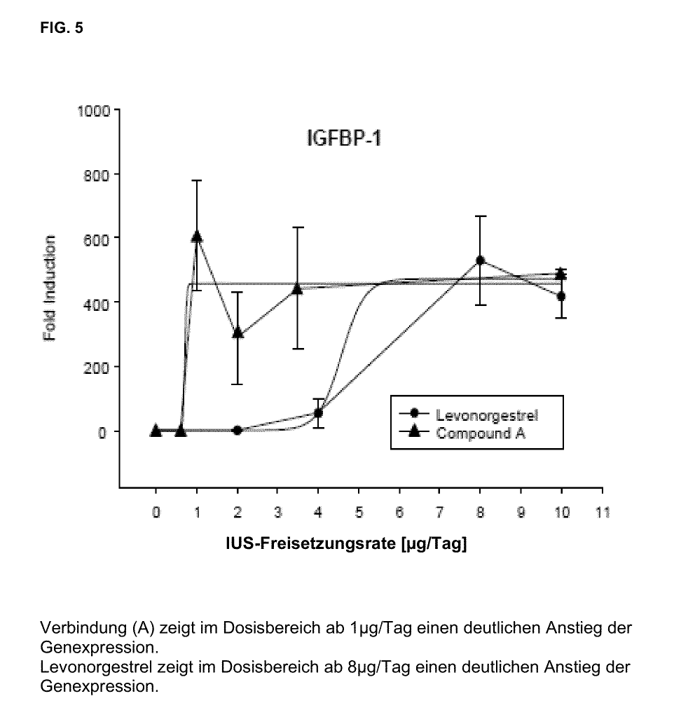

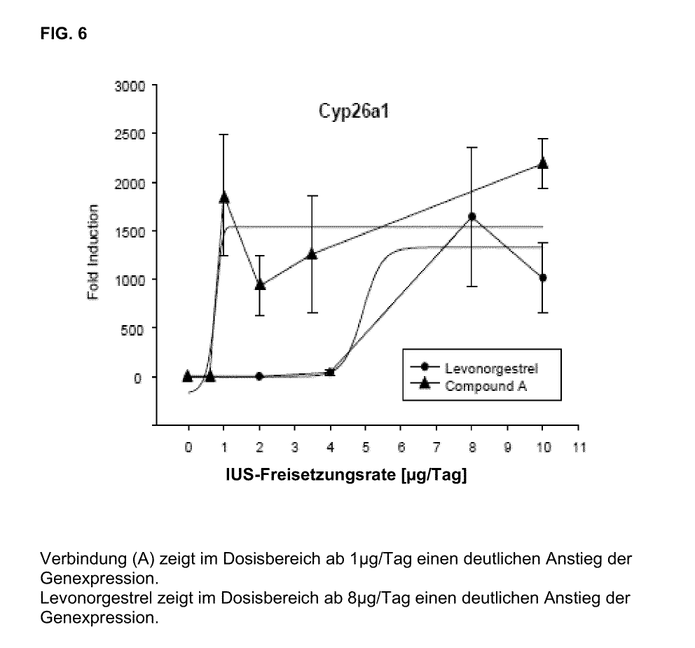

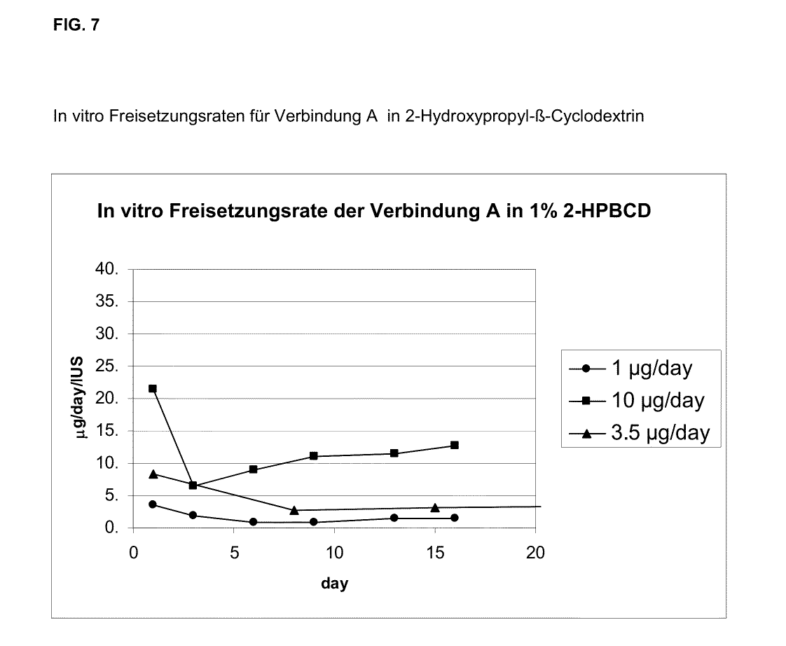

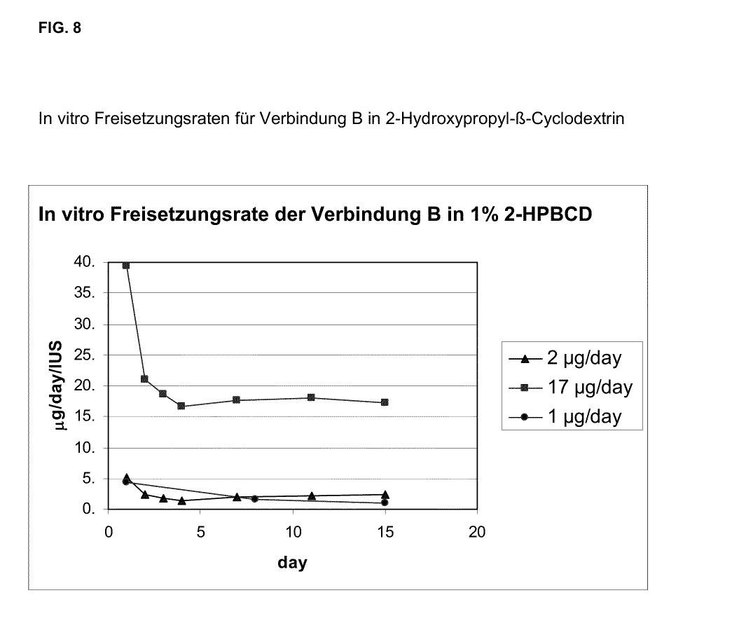

The in-vitro release rates stated in FIG. 4/4 were determined for a rod enveloped by a PTFPMS membrane.

1. (canceled)

2. Intrauterine system comprising 18-methyl-15β,16β-methylene-19-nor-20-spirox-4-en-3-one of the general formula (1) 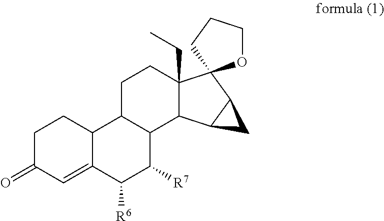
wherein R⁶ and R⁷ are a hydrogen atom or together an α-methylene group.

3. Intrauterine system according to claim 2, comprising 18-methyl-15β,16β-methylene-19-nor-20-spirox-4-en-3-one.

4. Intrauterine system according to claim 2, comprising 18-methyl-6α,7α,15β,16β-dimethylene-19-nor-20-spirox-4-en-3-one.

5. Intrauterine system according to claim 3, characterized in that a daily dose of 1-200 μg of 18-methyl-15β,16β-methylene-19-nor-20-spirox-4-en-3-one is released from the system.

6. Intrauterine system according to claim 5, characterized in that a daily dose of 1-100 μg of 18-methyl-15β,16β-methylene-19-nor-20-spirox-4-en-3-one is released from the system.

7. Intrauterine system according to claim 6, characterized in that a daily dose of 2-50 μg of 18-methyl-15β,16β-methylene-19-nor-20-spirox-4-en-3-one is released from the system.

8. Intrauterine system according to claim 4, characterized in that a daily dose of 2-500 μg of 18-methyl-6α,7α,15β,16β-dimethylene-19-nor-20-spirox-4-en-3-one is released from the system.

9. Intrauterine system according to claim 8, characterized in that a daily dose of 2-200 μg of 18-methyl-6α,7α,15β,16β-dimethylene-19-nor-20-spirox-4-en-3-one is released from the system.

10. Intrauterine system according to claim 9, characterized in that a daily dose of 5-100 μg of 18-methyl-6α,7α,15β,16β-dimethylene-19-nor-20-spirox-4-en-3-one is released from the system.

11-12. (canceled)

13. A method for contraception or gynaecological treatment, except for menorrhagia and other uterine haemorrhages, comprising the step of administering 18-methyl-15β,16β-methylene-19-nor-20-spirox-4-en-3-one to a patient in need thereof.
